(19) United States
(12) Patent Application Publication
(10) Pub. No.: US 2006/0106342 A1
(43) Pub. Date: May 18, 2006

(54) Injection applicator for a hypodermic syringe

(76) Inventor: Michael Cox, Woodlawn (CA)

Correspondence Address: Suzannah K. Sundby;Smith, Gambrell & Russell, LLP, Suite 800, 1850 M Street, N.W., Washington, DC, 20036, US

(21) Appl. No.: 11/067,614

(22) Filed: Feb. 28, 2005

Related U.S. Application Data
(60) Provisional application No. 60/583,037, filed on Jun. 28, 2004.

(51) Int. Cl. A61M 5/00

(52) U.S. Cl. 604/110

(57) ABSTRACT

There is provided an injection device comprising a barrel adapted for receiving a syringe assembly, a trigger assembly, tension spring means and stopping means. The trigger assembly comprises retractable blocking means for blocking the syringe assembly at a first axial point within the barrel and a trigger for retracting said blocking means from blocking the syringe along said first axial point along the barrel. The tension spring means has a barrel end mountable on the barrel and a syringe end mountable on the syringe assembly, whereby said tension spring means imparts an axial force to the syringe assembly when the syringe assembly is in a cocked position. The stopping means are for retaining the syringe assembly in an injected position within the barrel upon release of the syringe assembly from the cocked position followed by axial travel of the syringe assembly within the barrel under the influence of the axial force.

### FIELD OF INVENTION

This invention is directed to injection devices and particularly but not exclusively to an applicator for a hypodermic syringe.

### BACKGROUND OF THE INVENTION

The requirement for self, or home-administered injections by non-medical personnel is becoming more common. Some of the reasons for this need are increased prescriptions of injectable medicaments including hormones and vitamins. For example, medicaments such as insulin have to be administered frequently and often at irregular times. Also, in many cases a person requiring a injectable medicament may be incapacitated to some degree, for example, in cases of asthma or reactions to such things as allergens, insect stings, venom toxins or the like when an antidote, for example, atropine, adrenaline (epinephrine) or other medicament must be immediately injected.

Various systems have been proposed in the past for assisting with injections such as in those described in U.S. Pat. Nos. 4,787,891, 6,159,247, 6,077,247, 3,605,743, 2,047,010, 2,150,738, 2,664,086, and 4,067,334. However, these devices present one or more of the following disadvantages: are not simple and easy to use, are relatively complex and therefore expensive; require special syringe types, i.e. cannot be used with standard, unmodified, over-the-counter syringes; cannot be aspirated or the syringe contents monitored during the injection process; cannot be used as an aid for filling the syringe from an ampoule; do not permit variation of the dosage or type of medicament; are not re-usable; do not allow for the rate of infusion to be easily varied, monitored or controlled; or cannot be used for deep, intramuscular type injections.

There is a need for an improved, simple to use, inexpensive device to assist people in the use of commonly available disposable hypodermic syringes. Further, there is a need to overcome other disadvantages in the art.

### SUMMARY OF THE INVENTION

An object of the invention is to provide an injection device that alleviates totally or in part the drawbacks of the prior art injection devices.

Another object of the invention is to provide an applicator for a hypodermic syringe that assists those who have some difficulty or lack training in injecting either themselves or others.

Accordingly, there is provided an injection applicator comprising a barrel adapted for receiving a syringe assembly, a trigger assembly, tension spring means and stopping means. The trigger assembly comprises retractable blocking means for blocking the syringe assembly at a first axial point within the barrel and a trigger for retracting said blocking means from blocking the syringe along the first axial point along the barrel. The tension spring means has a barrel end mountable on the barrel and a syringe end mountable on the syringe assembly, whereby said tension spring means-imparts an axial force, for example, but not limiting to an axial pulling or pushing force to the syringe assembly when the syringe assembly is in a cocked position. The stopping means are for retaining the syringe assembly in a fired or injected position within the barrel upon release of the syringe assembly from the cocked position followed by axial travel of the syringe assembly within the barrel under the influence of said axial force. A method of using the above described injection device is also provided.

Also contemplated by the present invention is an injection applicator as defined above, wherein the trigger assembly further comprises a locking means that biases the blocking means from a non-blocking position toward a blocking position, whereby the non-blocking position corresponds to the blocking means being retracted by a suitable force applied to the trigger.

The present invention also provides an injection applicator as described above wherein the locking means comprises a leaf spring. Further, the trigger assembly may comprise a trigger and a sear.

The present invention also provides an injection applicator as described above wherein the tension spring means comprises one or more springs, for example, but not limited to metallic coils, elastic materials, rubber bands, or a combination thereof.

It is also contemplated that the injection applicator may further comprise a syringe assembly. The syringe assembly may comprise a plunger, a syringe body comprising at least one flange, a socket for receiving a screw-in needle, for example, but not limited to, of the luer lock type, or alternatively, the socket may receive a push-in needle. The syringe assembly may also comprise a needle.

The present invention also provides an injection applicator as described above wherein the barrel, trigger assembly and stopping means comprises a single molded part from one piece of material. Preferably, the material is a substantially clear, transparent material, for example, but not limited to a plastic. In specific embodiments, which are not meant to be limiting, the material may comprise acrylic, polystyrene (lucite), polycarbonate, or any other molded or injection molded plastic, or the like. In the event that barrel is made of a non-transparent material, preferably the barrel comprises at least one aperture or window permitting the contents of a syringe to be observed by a user during injection.

In an embodiment of the present invention, which is not meant to be limiting in any manner, there is provided an injection applicator as defined above wherein the trigger assembly comprises a trigger and sear having a roughly rectangular shape with three bends, the sear fitting through at least one slot in the barrel to engage a portion of a syringe when the syringe is disposed in the applicator, the sear retained in the slot by the action of a leaf spring overlapping a surface of the trigger, the trigger rotatable about a fulcrum when sufficient force is applied to a surface thereof. In an embodiment of the present invention, which is not meant to be limiting in any manner, the injection applicator comprises a one-piece trigger that incorporates the three functions of lever, axis and sear.

Also contemplated by the present invention is an injection applicator as defined above wherein the locking means comprises a spring, for example, but not limited to a tension or compression spring, or other suitable member positioned between the trigger and the barrel, and attached to each, such that a force is exerted on the trigger that constantly urges the sear into a blocking position.

The present invention also provides an injection applicator as defined above wherein the trigger assembly comprises a trigger and sear, and the trigger and sear are pivotably attached to the barrel such that the sear is normally biased toward a blocking position, and wherein application of an appropriate force to the trigger results in movement of the trigger about its pivot axis thereby relieving the sear from the blocking position, and permitting acceleration of a cocked syringe.

Also provided by the present invention is an injection applicator as defined above, wherein the trigger assembly, blocking means and barrel comprise a single continuous material and wherein, a force applied at or near the distal end of the trigger, directed about perpendicular to the plane thereof and in the general direction of the barrel, produces a lever force that is transmitted to a portion of the barrel comprising the blocking means and results in deflection of the blocking means outward of the plane of the barrel releasing a syringe when in the cocked position. For example, but not wishing to be limiting, the lever force may produce a torque that in turn affects the cantilever in a part of the barrel.

The present invention also provides an injection applicator as defined above and further comprising one or more of a filling attachment comprising an adapter and an ampoule holder, an adjustable spacer, a non-adjustable spacer, and a needle guide.

Also provided by the present invention is a kit comprising one or more injection applicators as defined above and optionally one or more: tension spring means, syringe assemblies, needles, syringes, filling attachments, spacers, needle guides, medicaments or other injectable substances, alcohol, cotton swabs, gauze pads or the like, band-aids, instructions, spare parts for the injection applicator or syringe assembly, or any combination thereof.

Advantageously, the injection device according to the invention provides an inexpensive, simple applicator for hypodermic syringes. Another advantage of the invention according to one of its embodiments is having the ability to control the depth of needle insertion. The applicator may be used to assist with shallow, subcutaneous type injections but also deeper injections, for example, but not limited to deep, intramuscular injections. It is also possible that the injection applicator may be used to deliver other types of injections, for example, but not limited to intravenous injections. The applicator allows for quick insertion of the needle and allows for a controlled infusion of the medicament, thereby minimizing pain or unpleasant sensations. Once the injection applicator is placed on the injection site, it may be operated without looking at it, making it easier to use by individuals with a fear of needles or those with impaired sight. The insertion of the needle can be performed, for example, using two fingers on one hand. Thus, it can be used by persons with impaired manual dexterity.

Yet another advantage according to a further embodiment, is the ability to ensure that a medicament ampoule is efficiently emptied into the syringe thereby minimizing waste, and also the ability to allow easy, accurate filling of the syringe.

Other advantages, objects and features of the present invention will be readily apparent to those skilled in the art from a review of the following detailed description of preferred embodiments in conjunction with the accompanying drawings.

This summary of the invention does not necessarily describe all features of the invention.

### BRIEF DESCRIPTION OF DRAWINGS

The following detailed description, given by way of example and not intended to limit the present invention solely thereto, will best be appreciated in conjunction with the accompanying drawings, wherein like reference numerals denote like elements and parts, where:

FIG. 1 is an isometric view of the applicator assembly 30 in accordance with one embodiment of the invention;

FIG. 1a is the back view of FIG. 1 showing the integral thumb rest 42 and a crosshatched area 45 where a window would be cut out if the body of the device were to be made from an opaque material;

FIG. 2 is an isometric illustration of a typical disposable hypodermic syringe assembly 10 commonly available over-the-counter;

FIG. 3 is an isometric view showing the applicator and syringe of FIGS. 1 and 2 assembled in the “cocked” position;

FIG. 4 is an isometric “exploded” view of FIG. 3;

FIG. 5 is an orthographic top view of FIG. 3;

FIG. 6 is a cross-sectional view of FIG. 5 taken along the dashed line in the direction of the arrows labeled FIG. 6 in FIG. 5;

FIG. 7 is a detailed view of the trigger assembly holding the syringe in the “cocked” position as shown in the area in the ellipse in the sectional view FIG. 6;

FIG. 8 is an orthographic top view of the applicator and syringe assembly in FIGS. 1 and 2, in the “injected” position;

FIG. 9 is a cross-sectional view of FIG. 8 taken along the dashed line in the direction of the arrows labeled FIG. 9, shown in FIG. 8;

FIG. 10 is a detailed view of the trigger assembly holding the syringe in the “injected” position as shown in the area in the ellipse in the sectional view FIG. 9;

FIG. 11 is an isometric view of the assembly of the applicator 30, syringe 10, filling attachment 80 and ampoule 82 in position ready for filling, according to a further embodiment of the invention;

FIG. 12 is an exploded view of the ampoule and filling attachment, according to the embodiment in FIG. 11;

FIG. 13 shows the filling attachment assembly 80 at approximately mid length adjustment;

FIG. 14 shows the adjustable spacer assembly 100 for controlling needle depth, according to a further embodiment of the invention;

FIG. 15 is an exploded view of FIG. 14.

FIG. 16 is an orthographic top view of the applicator, in accordance with another embodiment of the invention, showing a shorter barrel 40 with reduced diameter bore at the end 58 that forms the surface 51 which acts as the stopping means for the syringe assembly in the “injected” position;

FIG. 17 is a cross-sectional view of FIG. 16 taken along the dashed line in the direction of the arrows labeled FIG. 17, shown in FIG. 16;

FIG. 18 is an enlarged view of the area in the ellipse in the sectional view of FIG. 17 showing the syringe surface 20 in contact with the barrel surface 51, thus stopping further movement of the syringe.

FIG. 19 is an isometric view of the applicator assembly comprising a syringe in a cocked position, in accordance with an embodiment of the invention.

FIG. 20 is a rotated isometric view of the of the applicator assembly comprising a syringe in a fired position, in accordance with an embodiment of the invention.

FIGS. 21a-c depict partial, cross-sectional views of a barrel showing trigger assembly and blocking means including a trigger, and sear which is formed from a single continuous piece of material.

FIG. 22 is an exploded view of an embodiment of the applicator device comprising a syringe.

FIG. 23 is an isometric view of the applicator assembly comprising a syringe in a cocked position, in accordance with an embodiment of the invention.

FIG. 24 is an isometric view of the applicator assembly comprising a syringe in a fired position, in accordance with an embodiment of the invention.

FIG. 25 is a cross-sectional view of the applicator assembly comprising a syringe in a cocked position, in accordance with an embodiment of the invention.

FIG. 26 is a cross-sectional view of the applicator assembly comprising a syringe in a fired position, in accordance with an embodiment of the invention.

FIG. 27 is an isometric view of the applicator assembly comprising a syringe in a cocked position, in accordance with an embodiment of the invention.

FIG. 28 is an isometric view of the applicator assembly comprising a syringe in a fired position, in accordance with an embodiment of the invention.

FIG. 29 is a disassembled view of the applicator assembly and syringe, in accordance with an embodiment of the present invention.

FIG. 30 is a cross-sectional view of an embodiment of the injection applicator in a cocked position.

FIG. 31 is a top view of an embodiment of the injection applicator.

FIG. 32 is a cross-sectional view of an embodiment of the injection applicator in a fired position.

### DETAILED DESCRIPTION OF THE INVENTION

The following description is of a preferred embodiment.

Referring to the figures, and without wishing to be limiting in any manner, there is provided an injection device or applicator 30 comprising a barrel 40 adapted for receiving a syringe assembly 10, a trigger assembly 70, tension spring means 31 and stopping means 56. The trigger assembly comprises retractable blocking means 72 for blocking the syringe assembly at a first axial point within the barrel and a trigger for retracting said blocking means 72 from blocking the syringe assembly 10 along said first axial point along the barrel 40. The tension spring means 31 have a barrel end B mountable on the barrel and a syringe end S mountable on the syringe assembly 10, whereby said tension spring means 31 impart an axial pulling or pushing force to the syringe assembly 10 when the syringe assembly 10 is in a cocked position. The stopping means 56 are for retaining the syringe assembly in an injected position within the barrel 40 upon release of the syringe assembly 10 from the cocked position followed by axial travel of the syringe assembly 10 within the barrel 40 under the influence of said axial force.

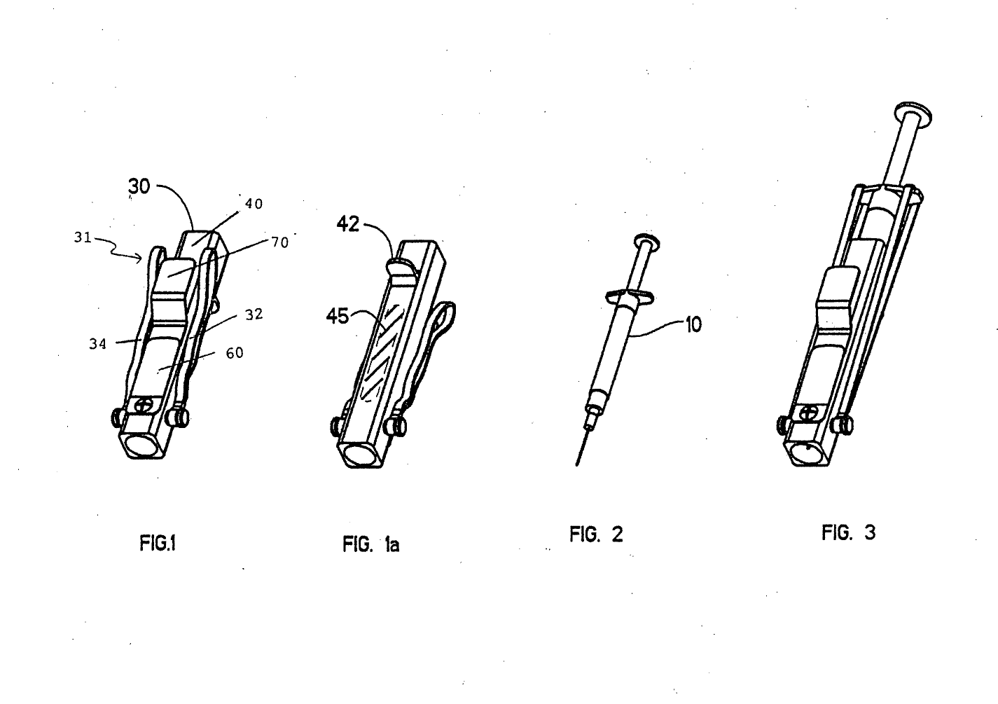

FIGS. 1-4 are isometric views of an applicator and a hypodermic syringe in accordance with an embodiment of the invention. Referring to FIGS. 1-4, the trigger assembly 70 is represented by a trigger and sear combination and the tension spring means 31 are represented by two identical tension springs 32 and 34. The trigger and sear combination comprises trigger 78 and blocking means or sear 72. According to the described embodiment, the applicator also comprises locking means 60, which, in conjunction with the trigger assembly 70 allow the blocking means or sear 72 to assume either a blocking or non-blocking position. In the described embodiment, which is not meant to be limiting in any manner, the locking means 60 is represented by a leaf spring.

In operation, the applicator 30 can be used with any syringe, for example, but not limited to those syringes commonly known in the art, or commercially available.

Further, the applicator 30 can be used with any commonly available, disposable, hypodermic syringe assembly generally designated 10. The hypodermic syringe 10 can be a typical commercially available type of assembly and comprises a plunger 12, syringe body 18, two flanges 14 and 16 on the body 18 and a flat surface 20 formed by the socket for the screw or push-in needle 22.

The present invention also contemplates an injection applicator that is specific for a particular type of syringe, for example, but not limited to a 1 mL, 2 mL, 3 mL, 5 mL, 10 mL, 50 mL syringe or the like. In a specific embodiment, which is not meant to be limiting in any manner, the injection applicator is specific for a syringe that is commercially available from Becton Dickenson, Terumo, or Kendall. However, other manufacturers are also contemplated.

In the embodiment described, which is not meant to be limiting in any manner, the applicator barrel 40 is tubular, but may have one or more flat surfaces 52 along its length in which there is a slot 54. Alternatively, if the applicator barrel does not comprise a flat surface, the slot 54 may be present in a curved surface of the barrel. Without wishing to be limiting in any manner, on a surface, preferably the diametrically opposite surface from 52 there is optionally a short, flat protrusion or thumbrest 42. Anchors 46, 48 for example, but not limited to posts protruding from each side are for anchoring the elastic bands or tension springs 32, 34. There is a means of securing the leaf spring 60 on surface 52. This is shown as an integral stake 44 that would pass through aperture 62 for a plastic injection molded barrel, which is a preferred method of construction, but this could be done by a screw, rivet or other means known to those skilled in the art. The barrel 40 is preferably made of a clear, transparent, stiff material such as a polycarbonate plastic or glass or it may be made of one or more different materials, for example but not limited to one or more metals. Injection molded plastic is preferred, but the part could also be made by machining, casting, extruding or other process as is known in the art. In the event that the barrel comprises a non-transparent material, preferably, it also comprises a cut-away section or a transparent section to permit the contents of the syringe to be viewed when in use.

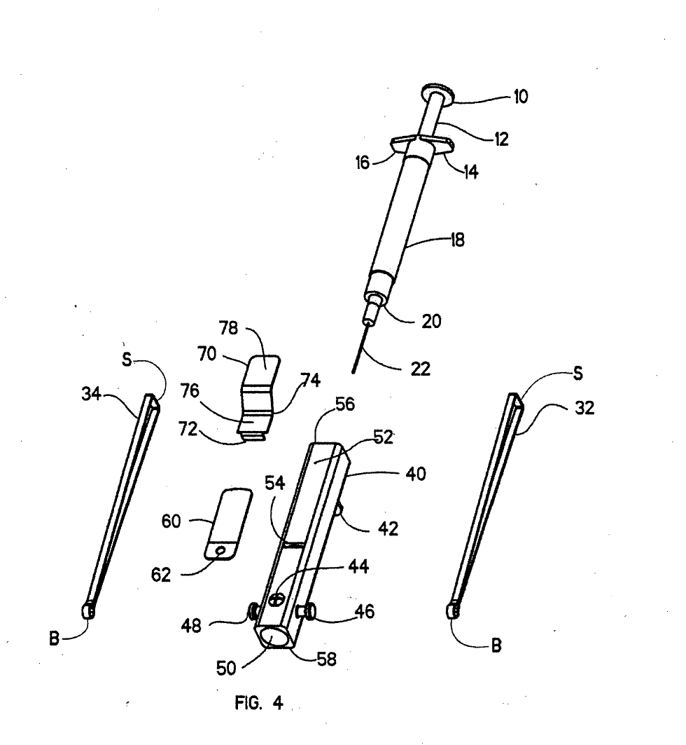

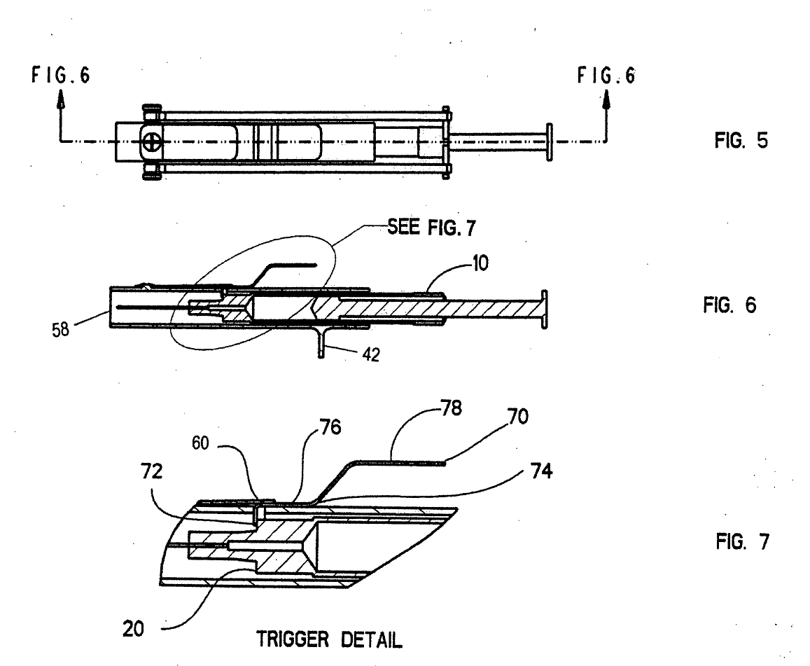

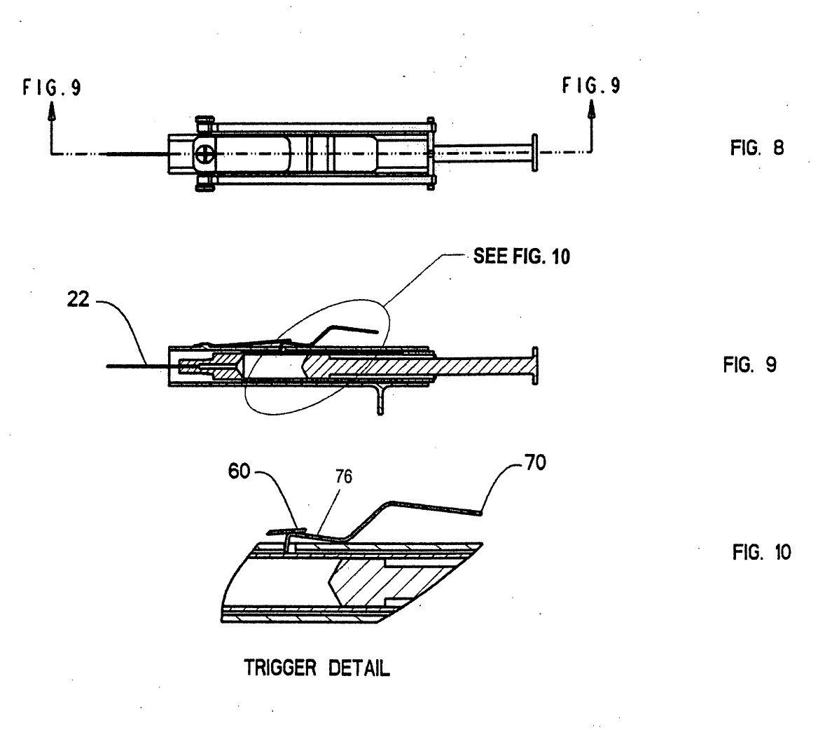

In an embodiment wherein the injector applicator comprises a leaf spring as locking means, the leaf spring 60 is a flat piece of stiff, resilient material, such as, but not limited to stainless steel, titanium, Phosphor Bronze, plastic or the like, and is provided with a means for attaching it to the barrel 40. In FIG. 4 this is shown as stake 44. However, a rivet or a screw may also be used. Similarly, it could also be attached by sliding the edges of the spring into one or more containment slots in the barrel, by adhesive, welding or other means. The leaf spring 60 could be formed of plastic and be molded integral with the barrel, or it could be formed by sheet metal stamping, as in the described embodiment.

According to one embodiment, which is not meant to be limiting in any manner, the trigger and sear assembly 70 is a single piece of sufficiently stiff material of roughly rectangular shape with three bends. The sear end 72 fits through the slot 54 in the barrel 40 with the flat 76 resting on surface 52 of the barrel. The sear 72 is retained in position in slot 54 by the action of the leaf spring 60, as the spring's surface overlaps the trigger surface 76. The trigger rotates against the force of the leaf spring 60 about a fulcrum formed by the radius of the bend at 74 when sufficient force is applied to surface 78 in the direction of the barrel. The trigger may be assembled or disassembled from the injection applicator, by deflecting the spring 60 enough for the sear 72 to be slipped out of, or in to, the slot 54. However, in normal operation, the trigger is unable to spontaneously disassemble itself from the injection applicator, as would be understood to a person of skill in the art.

In an alternate embodiment of the present invention, there is provided an injection applicator wherein the locking means 60 biases the trigger and sear combination into a blocking position to prevent the syringe from accelerating once in the cocked position. Any method known in the art that permits the trigger and sear to reversibly maintain this position is meant to be included by the present invention. For example, but not to be considered limiting in any manner, a spring member or the like may be positioned between the trigger and the barrel such that a force is exerted on the trigger that constantly urges the sear into a blocking position. The force of the spring member may be overcome by applying a greater counterforce in the opposite direction, the result being movement of the sear from the blocking position to the non-blocking position thereby permitting acceleration of a cocked syringe.

In still an alternate embodiment, the trigger may be pivotably attached to the barrel in a manner such that the sear is normally biased toward the blocking position. Application of an appropriate force to the trigger, for example, by a user requiring an injection results in movement of the trigger about it's pivot axis thereby relieving the sear from its blocking position, and permitting acceleration of a cocked syringe.

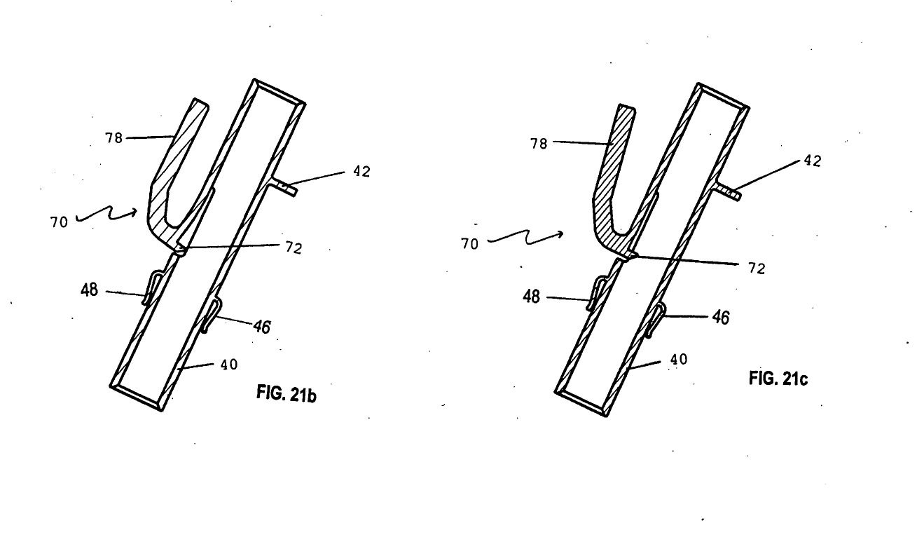

According to an alternate embodiment, which is not meant to be limiting in any manner, the barrel, trigger assembly and blocking means, for example, but not limited to the barrel 40, trigger 78, and sear 72 are molded from a single piece of material. As depicted in FIG. 19, there is shown an injection applicator wherein the applicator comprises a syringe and the applicator is cocked. FIG. 20 depicts an embodiment wherein the trigger has released the blocking means from the blocking position. In each of FIGS. 21a-c, there is shown a trigger assembly 70 and blocking means that are continuous with the barrel of the injection applicator. In such an embodiment, the trigger 78 may be designed, for example, but not limited to as shown in FIGS. 21a-c such that a force applied at or near the distal end of the trigger, directed about perpendicular to the plane thereof and generally in the direction of the barrel, produces a lever force that is transmitted to the portion of the barrel comprising the sear and results in deflection of the sear outward of the plane of the barrel releasing a syringe when in the cocked position. For example, but not wishing to be limiting, the lever force may produce a torque that in turn affects the cantilever in a part of the barrel.

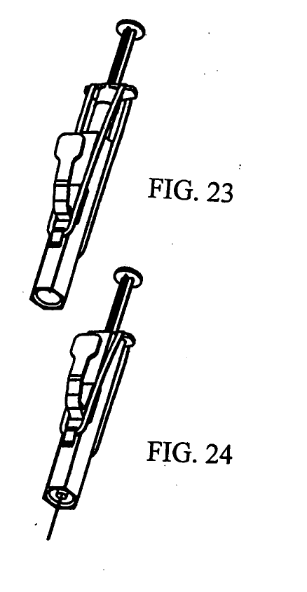

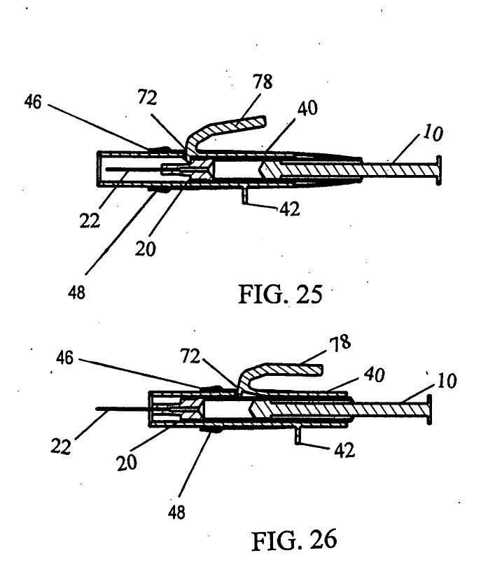

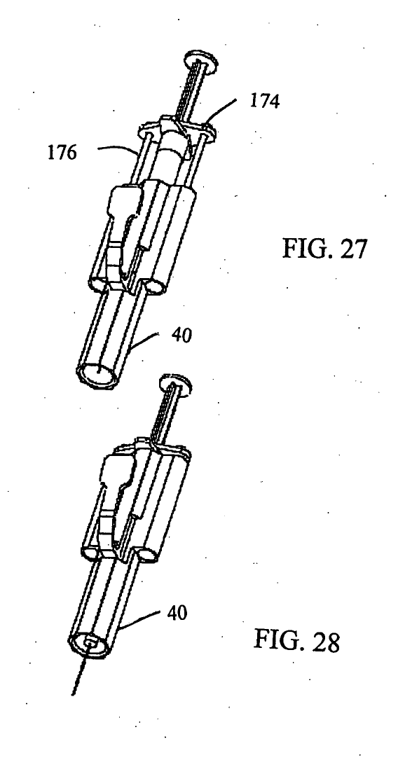

Shown in FIGS. 23 and 24 are further embodiments of the present invention in the cocked and fired position that are not meant to be limiting in any manner. FIGS. 25 and 26 represent cross-sectional views of the embodiments shown in FIGS. 23 and 24, respectively.

The elastic band tension springs 32 and 34 can be made from extrusions of sufficiently elastic material. They could also be helical or other type springs. In the embodiment shown, the elastic bands 32, 34 are attached to the barrel via an appropriate anchor, clip or as illustrated, but not wishing to be limiting, posts 46 and 48. It is also contemplated that each tension spring may be partially or completely hidden within a slot, tube, cylindrical cavity, or the like, for example, but not limited to telescopic tubing.

As depicted in FIGS. 23 and 24, the present invention further provides an injection applicator that comprises a single tension spring means, for example, but not limited to a helical spring or an elastic of a suitable material, for example, but not limited to rubber or the like. The single tension spring may attach to the barrel at a single position, for example, but not limited to an anchor or post as shown in FIG. 4 by either of numerals 46 or 48. Alternatively,it may be attached illustrated in FIGS. 23 and 24.

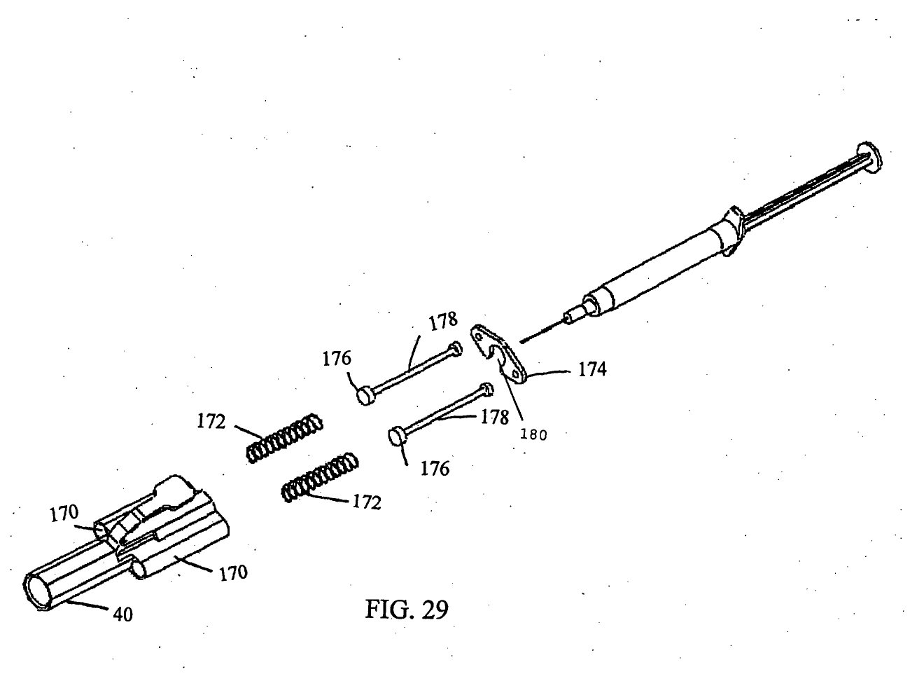

According to a further embodiment of the present invention, as shown in FIGS. 27-29 there is provided an injection applicator and syringe. In such an embodiment, which is not meant to be limiting in any manner, there is shown a barrel 40 that comprises two roughly cylindrical cavities 170 on opposite sides thereof. In each cavity 170 is provided a tension spring means, for example, but not limited to a compressible spring 172 that engages connecting link 178 at spring engagement member 176. Each connecting link also engages a portion of saddle member 174. A connecting link may comprise a rigid rod, hinged beam, flexible strap, or a combination thereof. Preferably, the connecting link is a rigid rod. In the embodiment shown, the walls defining cavities 170 are part of a single piece molding including the trigger, but they could be separate and able to swing about a mounting pivot on the barrel. Further, the saddle member may be a flat plate, or it may be more elaborate, for example, but not limited to including a catch for attaching it to the syringe. In the embodiment shown in FIG. 29, the saddle member 174 comprises a cut away portion 180 permitting engagement of the syringe in an appropriate manner, for example, as shown in FIG. 27.

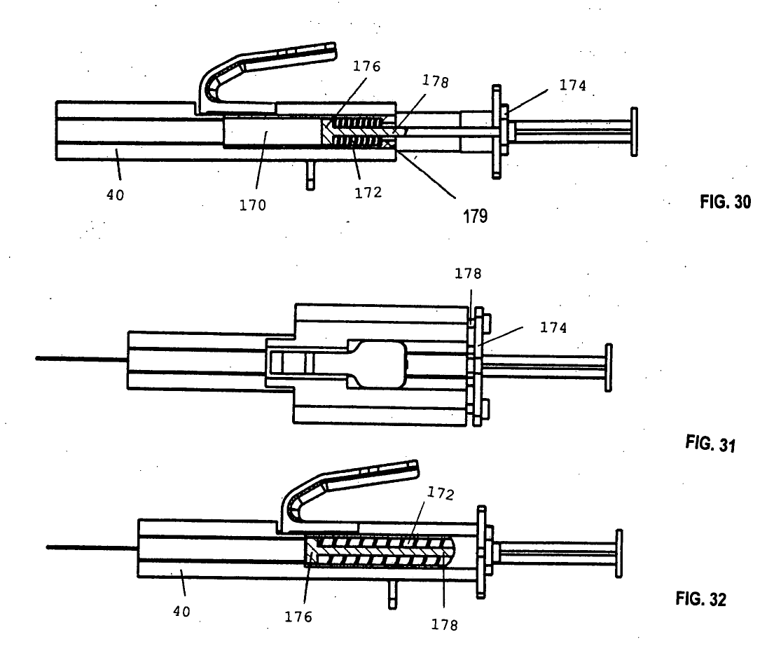

In the cocked position, the spring is compressed between spring engagement member 176 and a distal portion of the barrel 179 that forms cavity 170. As described previously, an appropriate force applied to the trigger releases the blocking means, and spring 172 exerts an axial pushing force that accelerates the syringe assembly toward the exit opening of the barrel. These embodiments are further depicted in FIGS. 30-32.

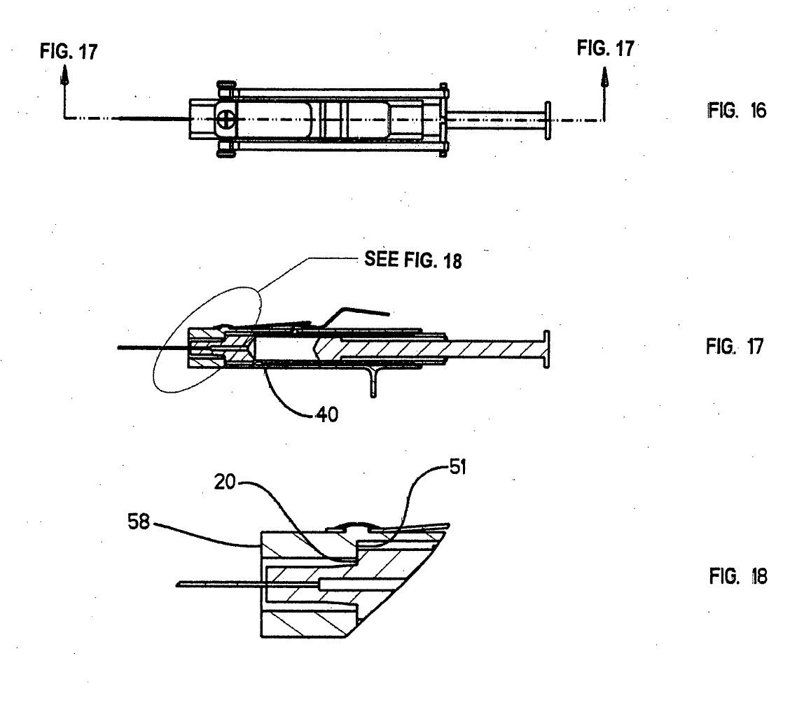

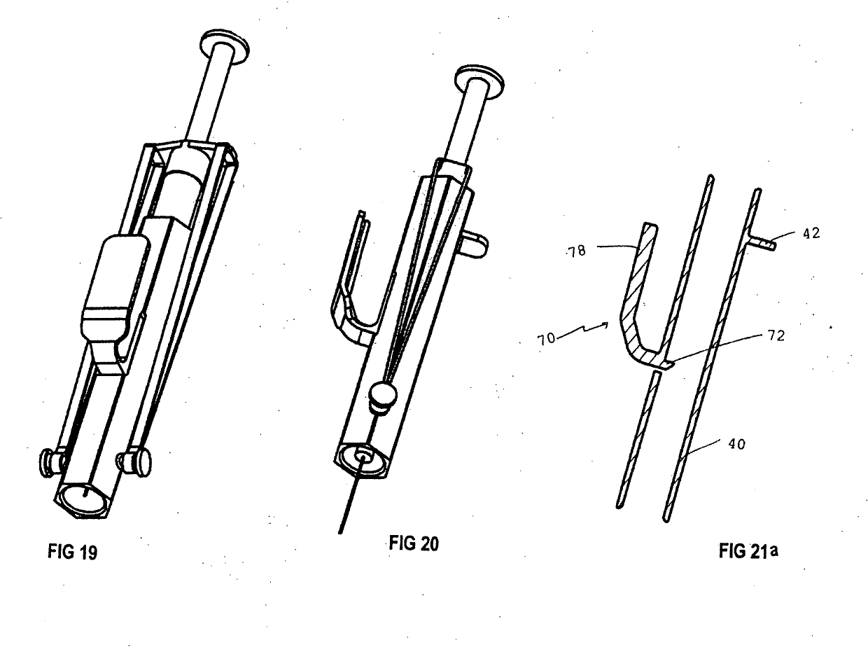

A method of using the applicator described above according to an embodiment of the invention is as follows. The method as described is not meant to be limiting in any manner. In operation, a syringe with the appropriate length of needle and filled with the specified dose of medicament is placed inside the bore 50 of the barrel 40 with the syringe surface, for example, but not limited to 20 resting on the sear 72 of the trigger 70. The tension springs 32 and 34 are then stretched over the syringe flanges 14 and 16 respectively, as in FIG. 3. The tension springs 32 and 34 force and hold the syringe assembly 10 hard against the sear 72, in a “cocked” position. The user places assembly 30 on to the injection site with the barrel surface 58 pressed against the skin. With the thumb resting on the thumb rest 42, the trigger lever 78 is pressed towards the barrel surface 52, preferably with the index finger of the same hand. This causes the trigger and sear assembly 70 to rotate about the fulcrum 74, thus pulling the sear 72 out of engagement with the syringe surface 20 (see for example FIGS. 6, 7 and 9). The syringe is pushed forward by the action of the tension springs until the syringe flanges 14 and 16 contact the barrel at surface 56 stopping further movement. This position of the syringe assembly within the barrel may be termed the “fired” or “injected” position. The needle is forced in to a preset depth, which is controlled by its length, the length of travel allowed, and the length of the barrel. It will be noted that, in this embodiment, stopping means 56 are represented by the surface 56 of the barrel opposite to the needle end. However, it will be apparent to those skilled in the art that stopping means 56 may be provided in a different manner, at a different site along the barrel, such as by adding a syringe stopping means, for example, but not limited to a physical block or a constriction site at or near the portion of the syringe that attaches to the needle, for example, near surface 58, to stop the forward movement of the syringe by contact with the syringe surface 20. This is shown in FIGS. 16, 17 and 18 wherein the stopping means is shown as a reduced diameter bore in the barrel where surface 20 of the syringe is brought in contact with surface 51 in the barrel. In this embodiment the barrel is shortened to ensure that the syringe flanges 14 and 16 cannot contact surface 56 before the movement is halted by the stopping means at 51.

Once in the “injected ” position, after the trigger 70 is released, it rolls back about the fulcrum 74 towards its original position, due to the force exerted by the leaf spring 60 onto its surface 76. Preferably, the sear 72 now grips the syringe body 18 tightly against the bore of the barrel under the influence of the leaf spring 60 (see for example FIG. 10). The syringe may now be held either at the flanges or by the barrel of the assembly with the other hand. The plunger can now be manipulated in the normal manner, for example, but not limiting to aspirate the syringe by pulling it back slightly and visually checking that there is no evidence of blood in the syringe before pushing it forward to deliver the dose. Aspiration is usually required for intramuscular type injections to ensure that the needle has not entered a blood vessel and the contents of the syringe usually have to be monitored at this stage. Similarly, for many applications, it is preferable that the syringe contents be visible in order to monitor the rate of delivery of the dose and to determine when the injection is completed. In order to allow monitoring, the barrel of the device could be made at least partially of transparent material, or a window could be cut in the barrel, in order to make the syringe body visible.

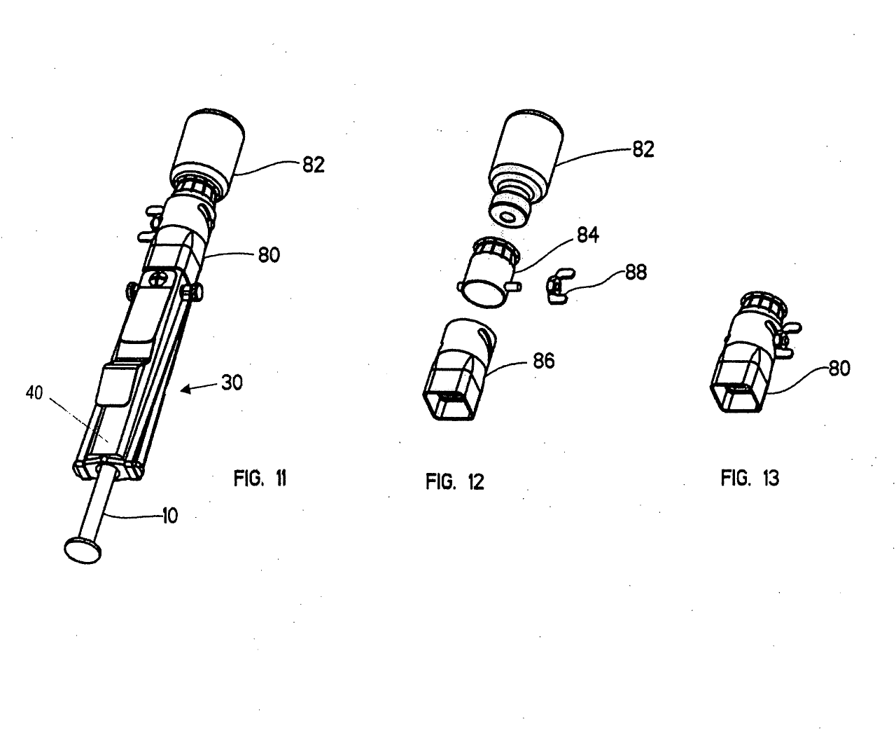

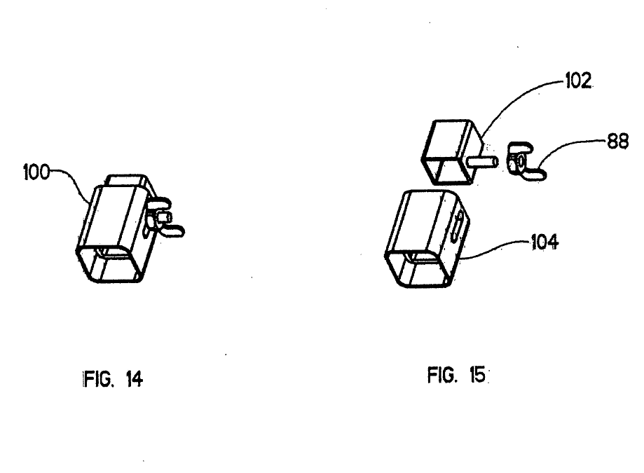

Referring now to FIGS. 11-13, there is shown a further embodiment of the invention, which is not meant to be limiting in any manner, wherein there is provided a filling attachment assembly 80 which comprises an adapter 86 that fits tightly over the end of the applicator barrel 40, a sliding ampoule holder 84 that may be positioned precisely along the length of the adapter 86 by rotating it and tightening a locking member for example, but not limited to a nut 88. Preferably, the holder 84 and the adapter 86 are injection molded from clear, transparent, strong plastic. However, other suitable materials may also be used. In the described embodiment, the ampoule holder end is formed into an annular spring by slotting it into a series of formed leaf springs. The annular spring grips the ampoule 82 firmly, exerting force against both the neck and shoulder of the ampoule, to hold it in position. The position of the needle within the ampoule may be set by adjusting the overall length of the attachment by rotating the ampule holder in relation to the adaptor when the two pieces are engaged, and then locking them in position with the nut 88, as shown in FIGS. 12 and 13.

According to another embodiment of the invention, an adjustable spacer 100 is used to control the depth of the needle by varying the length of the barrel. The spacer could be fitted on the distal end of the barrel, for example, the end the needle emerges during injection or it may be placed on the opposite side, on stopping means 56 as it will have the same effect there. The length of the spacer 100 may be adjustable by telescoping the two tubes 102 and 104 to the desired length then locking them in position with an appropriate locking member, for example, but not limited to nut 88.

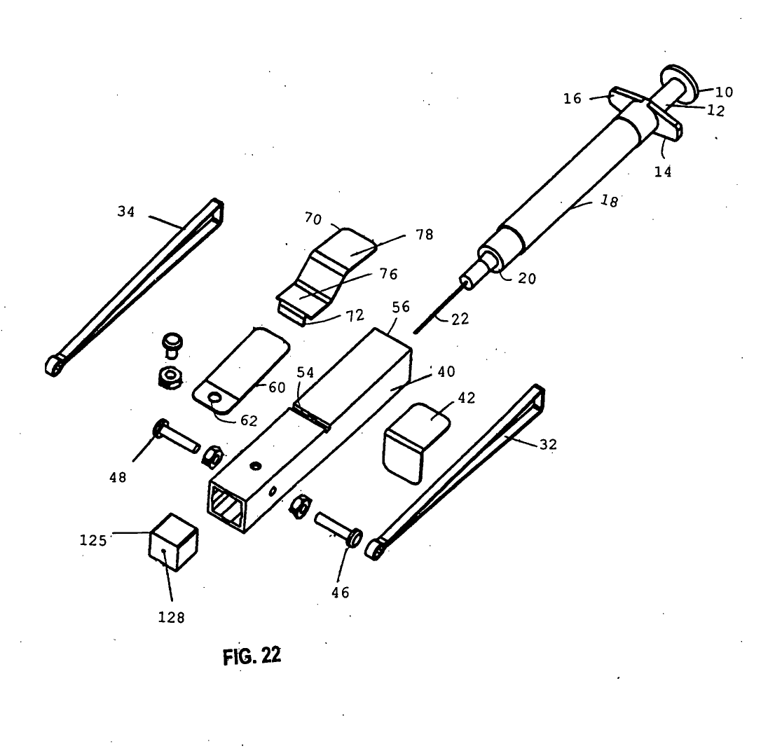

According to another embodiment, it is also contemplated that one or more non-adjustable spacers may be used in combination with the injection applicator. For example, but not wishing to be limiting in any manner, an appropriate spacer may be selected and attached to the needle end of the barrel or it could be placed closer to the syringe flange side of the barrel, or both when the applicator comprises a syringe. An example of such a spacer, which is not meant to be limiting in any manner is provided in FIG. 22 by spacer 125.

In an embodiment wherein the syringe spacer is attached to the needle end of the barrel, the spacer may further comprise a needle guide. Without wishing to be limiting in any manner the needle guide may comprise a channel within the spacer that directs the needle during its travel. Alternately, the needle guide may comprise an aperture in a surface of the spacer, for example, as shown by item 128 in FIG. 22. In this regard, it is also contemplated that the injection applicator itself may comprise a needle guide as described above without employing a spacer. The needle guide may be formed of a single material continuous with the barrel, for example, but not limited to by injection molding, as previously described. Any means known in the art may be employed to guide a needle. For example, but not wishing to be limiting in any manner, a needle guide may comprise a channel, slot, slit, aperture or the like that directs the needle during its travel from the cocked position to the injected or fired position.

The present invention also contemplates a kit comprising one or more injection applicators as described herein and throughout. The kit may further comprise one or more tension spring means, for example, but not limited to springs, elastics, elastic bands or the like, one or more syringes, one or more needles, a filling attachment, for example, but not limited to an adaptor, ampoule holder or both, one or more adjustable spacers, one or more non-adjustable spacers, one or more needle guides, a medicament or other injectable substance, alcohol, cotton swabs, gauze pads or the like, band-aids, instructions for using the injection applicator, instructions for using the medicament or other injectable substance, spare parts for the injection applicator or syringe assembly, or any combination thereof.

Without wishing to be considered limiting in any manner, the injection applicator as defined herein and throughout may be employed to administer an injection for a subject or patient, for example, but not limited to a human or animal subject or patient.

Although the present invention has been described in considerable detail with reference to certain preferred embodiments thereof, other versions are possible. Therefore, the spirit and scope of the appended claims should not be limited to the description of the preferred embodiments contained herein.

What is claimed is:

1. An injection applicator comprising:
a barrel adapted for receiving a syringe assembly;
a trigger assembly comprising
retractable blocking means for blocking the syringe assembly at a first axial point within the barrel; and
a trigger for retracting said blocking means from blocking the syringe along said first axial point along the barrel
tension spring means having a barrel end mountable on the barrel and a syringe end mountable on the syringe assembly, whereby said tension spring means imparts an axial force to the syringe assembly when the syringe assembly is in a cocked position, the barrel end is mounted on the barrel and the syringe end is mounted on the syringe assembly; and
stopping means for retaining the syringe assembly in an injected position within the barrel following release of the syringe assembly from the cocked position and axial travel of the syringe assembly within the barrel under the influence of said axial force.

2. The injection applicator of claim 1, wherein the trigger assembly further comprises a locking means that biases said blocking means from a non-blocking position toward a blocking position, whereby the non-blocking position corresponds to said blocking means being retracted by a suitable force applied to the trigger.

3. The injection applicator of claim 2, wherein said locking means comprises a leaf spring.

4. The injection applicator of claim 1, wherein the trigger assembly comprises a trigger and a sear.

5. The injection applicator of claim 1, wherein the tension spring means comprises one or more springs, elastic materials, rubber bands, or a combination thereof.

6. The injection applicator of claim 1, further comprising a syringe assembly.

7. The injection applicator of claim 6, wherein the syringe assembly comprises a plunger, syringe body comprising at least one flange, a socket for receiving a screw-in or push-in needle and optionally, a needle.

8. The injection applicator of claim 1, wherein the barrel, trigger assembly and stopping means comprises a molded, one-piece material.

9. The injection applicator of claim 8, wherein the material is a substantially clear, transparent material.

10. The injection applicator of claim 9, wherein the material comprises polycarbonate.

11. The injection applicator of claim 8, wherein the barrel comprises at least one aperture or window permitting the contents of a syringe to be observed by a user during injection.

12. The injection applicator of claim 1, wherein said barrel comprises at least one anchor for anchoring at least one tension spring means.

13. The injection applicator of claim 1, wherein said trigger assembly comprises a trigger and sear having a roughly rectangular shape with three bends, said sear fitting through at least one slot in the barrel to engage a portion of a syringe when said syringe is disposed in the applicator, said sear retained in said slot by the action of a leaf spring overlapping a surface of the trigger, said trigger rotatable about a fulcrum when sufficient force is applied to a surface of said trigger.

14. The injection applicator of claim 2, wherein said locking means comprises a spring member positioned between the trigger and the barrel such that a force is exerted on the trigger that constantly urges the sear into a blocking position.

15. The injection device of claim 1, wherein the trigger assembly comprises a trigger and sear, and said trigger and sear are pivotably attached to the barrel such that the sear is normally biased toward a blocking position, and wherein application of an appropriate force to the trigger results in movement of the trigger about its pivot axis thereby relieving the sear from the blocking position, and permitting acceleration of a cocked syringe.

16. The injection applicator of claim 1, wherein said trigger assembly, blocking means and barrel comprise a single continuous material and wherein, a force applied at or near the distal end of the trigger, directed about perpendicular to the plane thereof and in the general direction of the barrel, produces a lever force that is transmitted to a portion of the barrel comprising the blocking means and results in deflection of the blocking means outward of the plane of the barrel releasing a syringe when in the cocked position.

17. The injection applicator of claim 1, further comprising a filling attachment comprising an adapter and an ampoule holder.

18. The injection applicator of claim 1, further comprising an adjustable spacer, a non-adjustable spacer, or both.

19. The injection applicator of claim 1, further comprising a needle guide.

20. A kit comprising one or more injection applicators as defined by claim 1 and optionally one or more:
tension spring means, syringe assemblies, needles, syringes, filling attachments, spacers, needle guides, medicaments or other injectable substances, alcohol, cotton swabs, gauze pads or the like, band-aids, instructions, spare parts for said injection applicator or syringe assembly, or any combination thereof.
